(19) United States
(12) Patent Application Publication
(10) Pub. No.: US 2007/0142323 A1
(43) Pub. Date: Jun. 21, 2007

(54) Oligosaccharides, preparation method and use thereof, and pharmaceutical compositions containing same

(75) Inventors: Christian Viskov, Ris Orangis (FR); Pierre Mourier, Charenton Le Pont (FR)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: Aventis Pharma S.A., Antony (FR)

(21) Appl. No.: 11/508,800

(22) Filed: Aug. 23, 2006

Related U.S. Application Data
(63) Continuation of application No. PCT/FR05/00431, filed on Feb. 23, 2005.

(30) Foreign Application Priority Data
Feb. 24, 2004 (FR) 0401810

(51) Int. Cl. A61K 31/727; C08B 37/10; C12P 19/28

(52) U.S. Cl. 514/56; 435/85; 536/21

(57) ABSTRACT

The invention relates to oligosaccharides, the preparation method and use thereof, and pharmaceutical compositions containing same. More specifically, the invention relates to oligosaccharides which can be used for the treatment of cancer and, in particular, to prevent and inhibit the formation of metastases. The inventive oligosaccharides can be used, for example, during early breast, lung, prostate, colon or pancreatic cancer. The oligosaccharides can be administered subcutaneously, orally or intravenously. Moreover, said oligosaccharides can be used alone or together with other anticancer agents, e.g. cytotoxics such as docetaxel or paclitaxel.

The present invention relates to novel chemical compounds, particularly novel oligosaccharides, to the process for preparing them, to their use and to pharmaceutical compositions containing them. These oligosaccharides are useful for treating cancer, in particular for preventing and inhibiting the formation of metastases.

More particularly, according to a first aspect, the invention relates to a process for depolymerizing a polysaccharide which originally has anti-thrombotic properties.

Processes for depolymerizing polysaccharides with anti-thrombotic properties are known. Common aspects of these processes are:
aiming to obtain oligosaccharides of lower average molecular mass in order to limit the side effects which occur when the starting polysaccharides are used as medicinal products;
maintaining a satisfactory anti-thrombotic activity after depolymerization.

Commercial polysaccharides with anti-thrombotic properties, such as heparin or low-molecular-weight heparins such as enoxaparin, tinzaparin, or fragmin, are all heparanase inhibitors.

Under normal physiological conditions, cells express the enzyme heparanase. This enzyme makes it possible to indirectly regulate mitogenesis, neovascularization and tissue repair. One of the mechanisms of action of heparanase is to cleave heparan sulphate proteoglycan (HSPG). This glycosaminoglycan is present at the surface of endothelial cells and ensures cohesion of the basal membrane (extracellular matrix). Cleavage of heparan sulphate proteoglycan results in the release of growth factors such as FGF2. The release of growth factors is necessary for mitogenesis and angiogenesis. However, this is not sufficient to trigger these biological mechanisms; it is necessary for FGF2 to bind to a glycosaminoglycan in order to generate an allosteric modification of the protein and to promote its interaction with its receptor. In fact, through the cleavage of HSPG, heparanase generates heparan sulphate fragments which will bind to FGF2 and promote the interaction with its receptors and thus induce the biological mechanisms mentioned above. The cleavage of HSPG in the extracellular matrix and the destructuring of capillaries enable cellular extravastion.

Heparanase is overexpressed by tumour cells and therefore promotes metastases and neovascularization thereof. These phenomena are essential for the propagation and survival of cancerous tumours.

Heparin, a glycosaminoglycan structurally similar to heparan sulphate, is known to be a potent inhibitor of heparanase. This effect has for a long time been attributed to the presence of the specific ATIII-binding sequence. In fact, this sequence, although present to a lesser degree in heparan sulphate, is common to these two glycosaminoglycans. The heparanase cleavage zone is represented below: 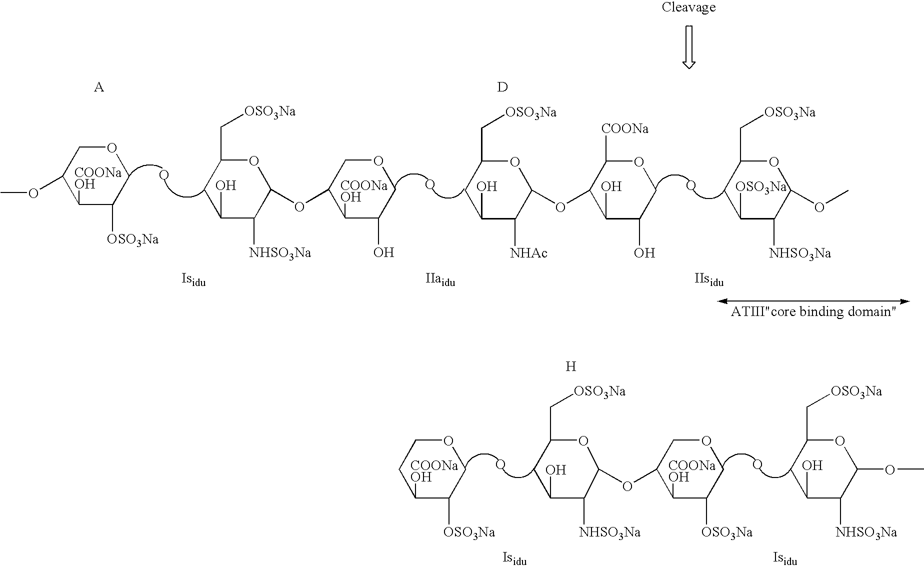
The point of cleavage by this α-endoglycosidase is located at the centre of the minimum ATIII-binding sequence. It may therefore be considered that the anti-thrombotic and heparanase-inhibiting properties are closely linked (in fact, heparin is a competitive substrate for heparan sulphate). As a result, it is difficult to use this glycosaminoglycan as an anti-metastatic. In fact, its strong anticoagulant properties limit its therapeutic margin and induce serious side effects such as severe haemorrhaging. Furthermore, repeated injection of heparin can, in certain cases, cause thrombocytopenia resulting in a fatal outcome (immunological reaction related to the association between heparin and platelet factor 4 (PF4)).

There are few documents which highlight the links between the structure of oligosaccharides and their anti-heparanase properties, in correlation with an anti-metastatic activity. Thus, Bitan et al. (Isr. J. Med. Sci. 1995; 31: 106-118) specifies the structural conditions required for the inhibition of pulmonary melanoma colonization by heparanase-inhibiting heparin species: heparanase is inhibited effectively by heparin fragments containing 16 or more sugars (summary; FIG. 2, p. 110; FIG. 3, p. 111; p. 116, right-hand column, 2nd sentence). Hexasaccharides are described as poor heparanase inhibitors (FIG. 8, p. 115). In addition, it is said that the inhibition of heparanase is only possible with molecules having a molecular mass greater than or equal to at least 4000 daltons (summary: p. 116, right-hand column, 4th sentence). However, the method for determining the heparanase inhibition is an indirect method, since it consists in evaluating the ability of cells to degrade the extracellular matrix in the presence of the various test products (p. 108; right-hand column, 2nd paragraph “Degradation of Sulfated Proteoglycans”). It is not therefore specific for heparanase. In addition, all of the products described are obtained by a method for cleaving heparin chemically (nitrous acid) (p. 108; left-hand column, 2nd paragraph “Heparin-Derived Oligosaccharides”).

See also:

Vlodavski I, et al. Modulation of neovascularization and metastasis by species of heparin, Heparin and related Polysaccharides, D. A. Lane, et al., Editor, Plenum Press, New York, 1992;
Parish C R, et al. Evidence that sulphated polysaccharides inhibit tumor metastasis by blocking tumor-cell-derived heparanases, Int. J. Cancer 40: 511-518, 1987.

At this time, there is a considerable need for anti-metastatic compounds, for which there is no commercially acceptable solution. The heparanase-inhibiting polysaccharides and oligosaccharides currently known are derived directly from natural sources (heparin) or from processes which are more or less difficult to implement (some low molecular weight heparins) and exhibit a marked anti-thrombotic component which is not compatible with anticancer treatments, in particular when the patient to be treated is at risk haemorrhaging.

One of the current problems is therefore to obtain a product exhibiting significant anti-heparanase activity, essentially free of anti-thrombotic activity, via a simple and reproducible process.

To this end, and surprisingly, it has been found that a novel process for depolymerizing a polysaccharide which originally has anti-thrombotic properties, in which the polysaccharide is depolymerized with heparinase 1 until its anti-thrombotic activity, due in particular to the inhibition of factors Xa and IIa, is essentially extinguished (<35 IU/mg), makes it possible to obtain a product which conserves significant anti-heparanase activity.

This process therefore constitutes an effective means for obtaining anti-heparanase-site-enriched products of the polysaccharide, while at the same time eliminating its anti-thrombotic component. The process is more advantageously used when the depolymerization of the polysaccharide is pursued until its anti-thrombotic activity is less than 20 IU/mg.

More particularly, the depolymerization is pursued until an average molecular mass of less than 5000 Da, preferably less than 3000 Da, is attained.

Against all expectations, the product obtained by this process, which is simple to implement, contains in particular hexasaccharides which are good heparanase inhibitors. In addition, these hexasaccharides have an average molecular mass considerably less than 4000 daltons, since it is generally between 1000 and 2000 daltons.

The polysaccharide is preferably a heparin.

The depolymerization is advantageously pursued until the hexasaccharide fraction mixture is essentially free of sulphated hexasaccharides ΔIs-Is_{id}-Is_{id} and ΔIs-Is_{id}-IIs_{glu}.

Enzymes are normally used under “physiological” conditions, i.e. under the conditions under which they normally function in vivo in the organisms from which they are extracted (in particular: pH, temperature, ionic strength, possibly physical cofactors (light, etc.) or chemical cofactors (coenzymes, etc.)). Most enzymes can be commonly used at temperature above their physiological temperature, for example 45-50° C. In our situation, and against all expectations, it was observed that the depolymerization can still take place at a temperature of preferably between 10 and 20° C., in particular 16° C., under acceptable conditions of selectivity and of kinetics, thus preserving as well as possible the heparanase-inhibiting compounds formed during the depolymerization reaction. In addition, this makes it possible to limit the final concentration of heparinase 1 in the reaction medium at the end of the reaction. In fact, carrying out the reaction at a temperature below the optimal reaction temperature for heparinase 1, which can be around 25-45° C., makes it possible to avoid an excessive number of additions of enzyme in the course of the reaction. Enzyme is usually added when a drop in reaction kinetics, other than due to substrate depletion, is observed. Consequently, the use of a relatively low reaction temperature makes it possible indirectly to facilitate a possible subsequent purification step, in particular due to the limited presence of enzyme. The depolymerization can therefore, as a result, be carried out at a temperature of between 5 and 40° C., preferably between 10 and 20° C.

In order to remove possible low molecular weight oligosaccharides which have formed during the depolymerization, in particular disaccharides and tetrasaccharides, the process according to the invention is advantageously pursued by means of a step in which the product of depolymerization of the polysaccharide is purified by gel permeation chromatography (GPC) at a pH below 8 and above 5.

The process according to the invention advantageously comprises a subsequent step of purification by high performance liquid chromatography (HPLC) in which a stationary phase, for example a silica, is a reverse phase which is (i) C18-grafted and (ii) grafted with cetyl trimethylammonium (CTA-SAX).

The process also comprises a first desalification step, advantageously comprising the use of a mobile phase containing an electrolyte in aqueous solution, said electrolyte preferably being essentially transparent between 200 and 250 nm. Acceptable electrolytes comprise NaCl, but for use with a UV detector between 200-250 nM, it is preferable to use perchlorates, methanesulphonates or phosphates of alkali metals such as Na. An acceptable stationary phase for the first desalification step is an anion exchange resin. A particularly preferred resin is a Sepharose Q® resin.

The process can also comprise a second desalification step, preferably using a molecular exclusion gel, for example and preferably of the Sephadex G10® type.

Another solution for detecting the products according to the invention in the fractions collected on exiting the HPLC column may optionally consist of the use of a defractometer.

Other acceptable desalification techniques include the use of osmotic techniques, for example using polymer membranes.

According to a second aspect, the invention relates to products obtained by a process in accordance with its first aspect.

Petitou et al. in J. Biol. Chem. (1988), 263(18), 8685-8690 disclose a hexasaccharide of formula ΔIs-IIa_{idu}-IIs_{glu}, in the form of sodium salt, isolated from the product obtained by a process of partial depolymerization of heparin with heparanase I. This product is described as exhibiting no anti-thrombotic activity. No other property of this product is demonstrated.

According to a third aspect, the invention relates to products of formula (I) 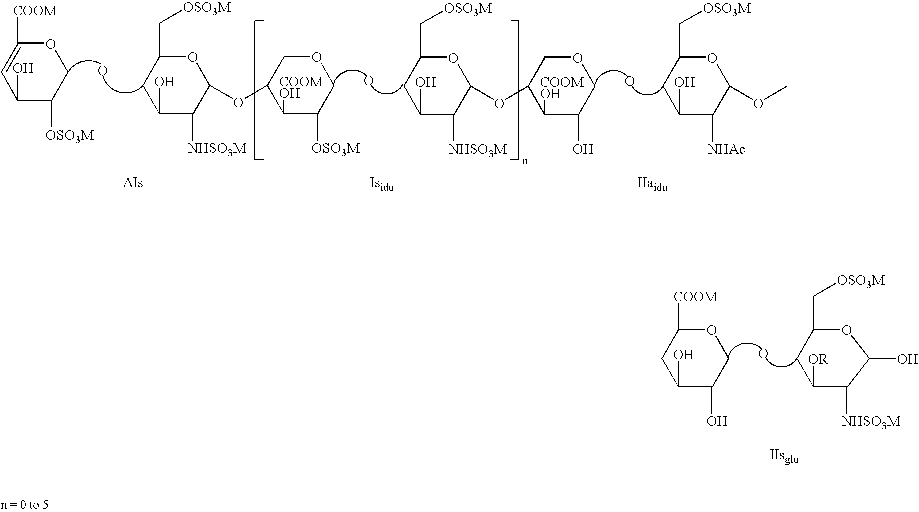
in which:

R is chosen from H and SO₃M, and

M is chosen from H, Li, Na and K;

with the exception of the product for which n=0, R=SO₃M and M=Na.

Unexpectedly, it has been observed that the products in accordance with the third aspect of the invention exhibit better physicochemical properties when M is chosen from Li, Na and K, preferably Na. In particular, the solubility and the stability are improved.

Preferred products of formula (I) are those for which n=0.

According to a fourth aspect, the invention relates to hexasaccharides.

A product of formula (Ia) below: 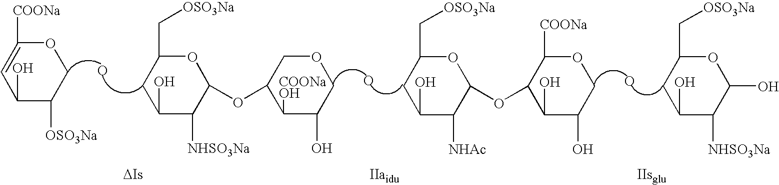
is in accordance with the invention according to another fourth aspect.

A product of formula (Ib) below: 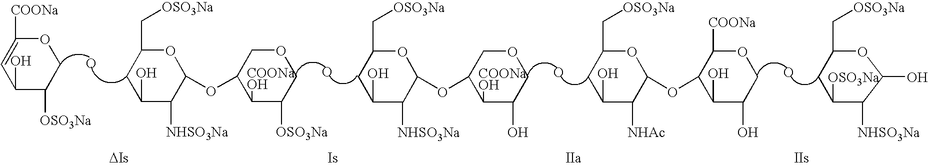
is in accordance with the invention according to another fourth aspect.

A product of formula (Ic) below: 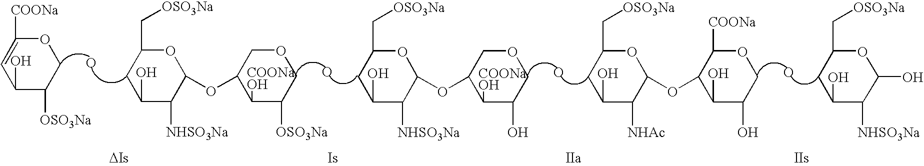
is in accordance with the invention according to another fourth aspect.

A product of formula (Id) below: 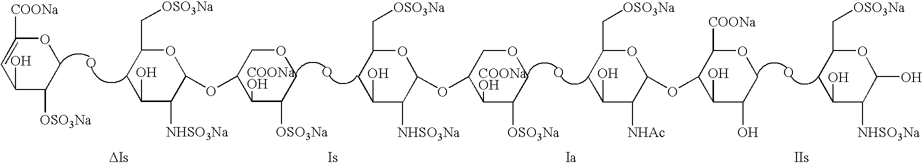
is in accordance with the invention according to another fourth aspect.

A product of formula (Ie) below: 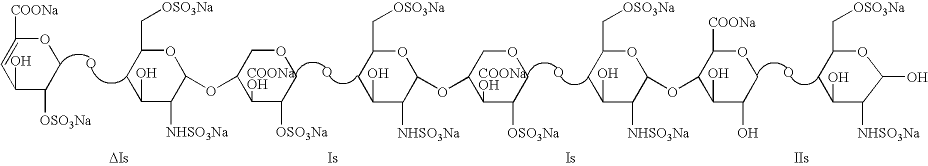
is in accordance with the invention according to another fourth aspect.

A product of formula (If) below: 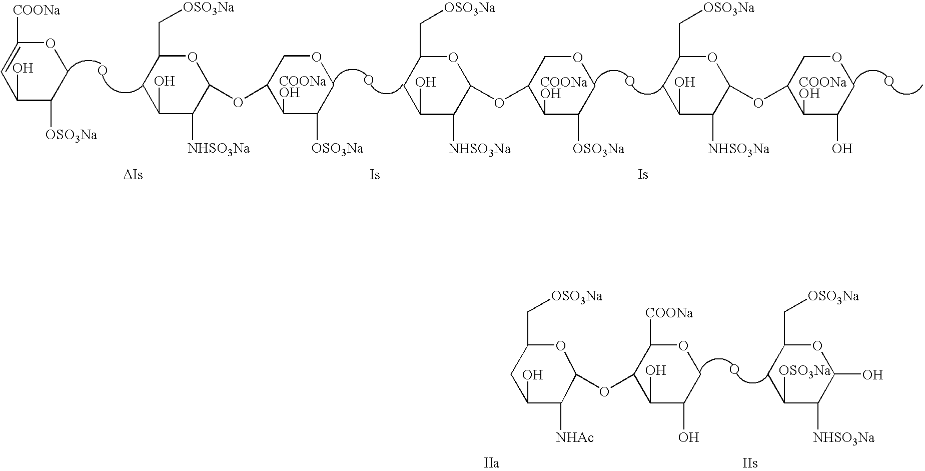
is in accordance with the invention according to another fourth aspect.

A product of formula (Ig) below: 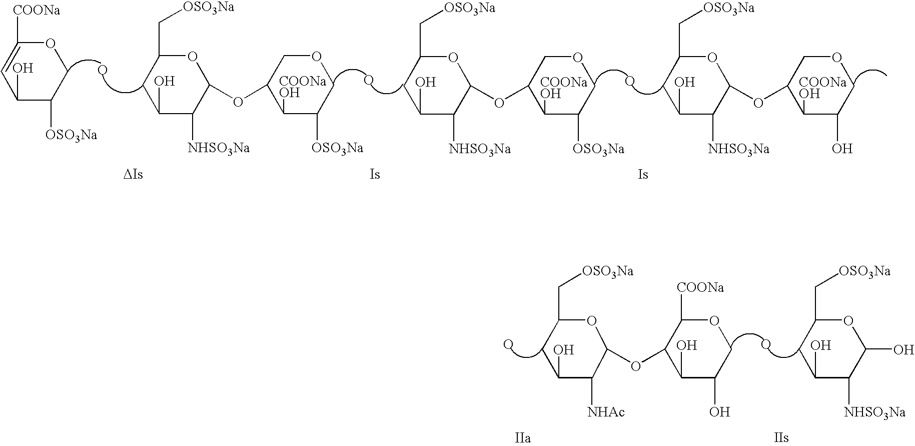
is in accordance with the invention according to another fourth aspect.

A product of formula (Ih) below: 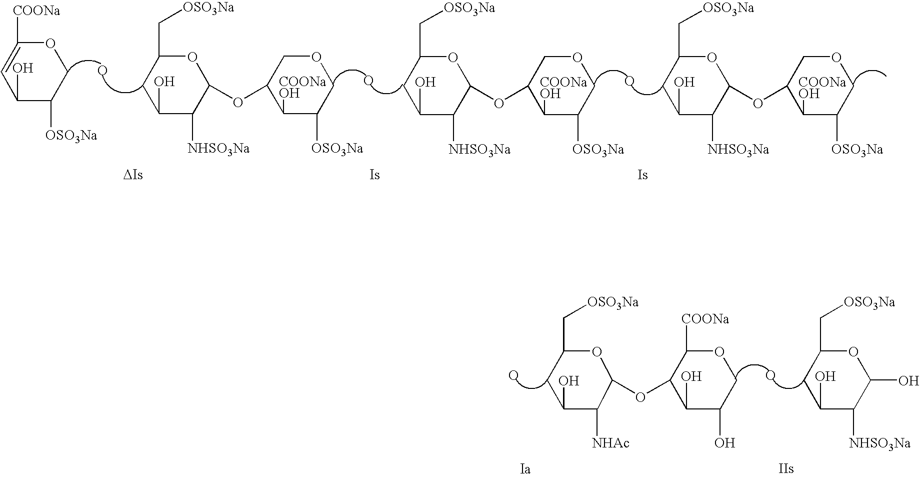
is in accordance with the invention according to another fourth aspect.

A product of formula (Ij) below: 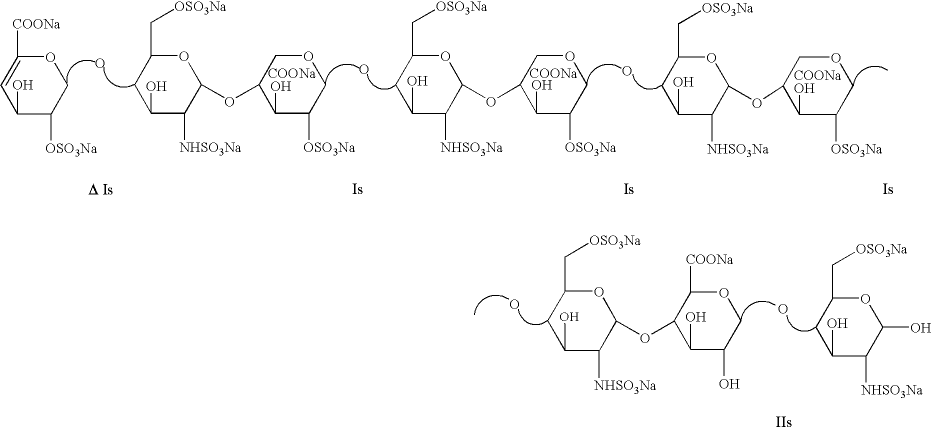
is in accordance with the invention according to another fourth aspect.

A product of formula (Ik) below: 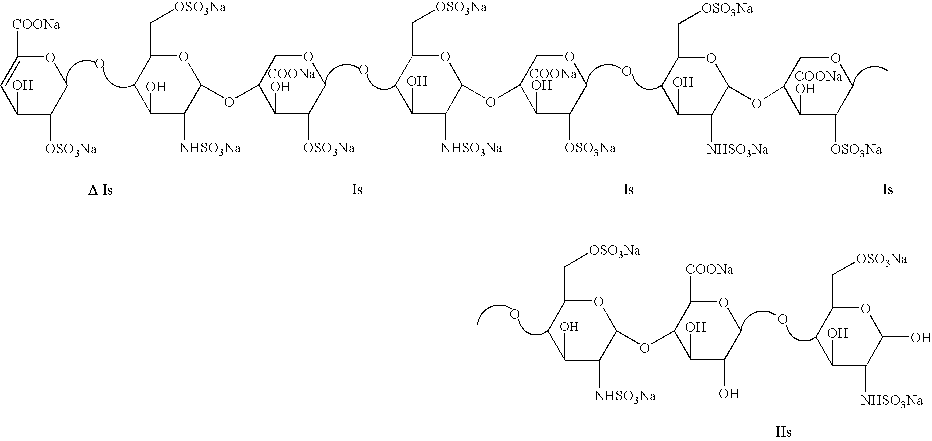
is in accordance with the invention according to another fourth aspect.

A product of formula (Im) below: 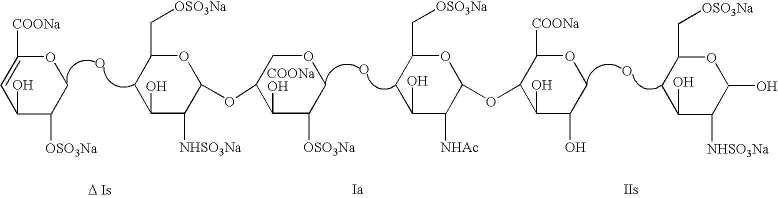
is in accordance with the invention according to another fourth aspect.

According to a fifth aspect, the invention relates to the use of a product according to any one of the second to fourth aspects, for modulating cell proliferation, in particular related to cancer, in particular breast cancer, lung cancer, prostate cancer, colon cancer or pancreatic cancer.

Use of a product according to the fifth aspect of the invention is particularly advantageous when the cell proliferation is related to a metastatic process, and also when the use is effected at an early stage of the disease.

Use of a product according to the fifth aspect of the invention is particularly advantageous in combination with a second anticancer, preferably cytotoxic, product.

A second anticancer product is advantageously chosen from platinum derivatives such as cisplatin or oxaliplatin, taxoids such as docetaxel or paclitaxel, purine base or pyrimidine base derivatives such as 5-FU, capecitabine or gemcitabine, vincas such as vincristine or vinblastine, mustards, condensed aromatic heterocycles such as staurosporine, ellipticine or camptothecins such as irinotecan, topotecan, combretastatins such as CA4P, and colchicine derivatives such as colchinol phosphate.

The second anticancer product is preferably docetaxel, oxaliplatin or irinotecan.

When the inhibition of heparanase by various commercial low molecular weight heparins is studied, it becomes evident that they all inhibit heparanase (enoxaparin, tinzaparin, fragmin, etc.). However, we were able to observe that a new ultra low molecular weight heparin (ULMWH) (WO 02/08295; and international application PCT/FR03/02960, Publication No. WO 04/033503) does not inhibit heparanase even though it comprises more sequences with affinity for ATIII than enoxaparin. Consequently, there is here an incoherence with respect to the theory stated above.

The process used according to the invention results in particular in the formation of a hexasaccharide, which is IsoATIII, of structure Is-IIa-IIs, below:
Hexasaccharide Is-IIa-IIs (Iso ATIII):

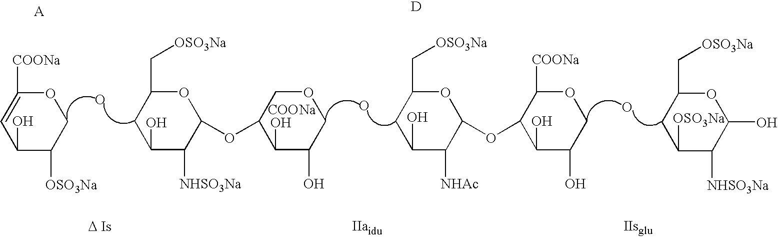

The results below show that this hexasaccharide Iso ATIII is a very good heparanase inhibitor. It also has the considerable advantage of having no affinity for ATIII and, consequently, of being devoid of anti-thrombotic activity. The major advantage of this invention is the separating of the anti-thrombotic properties of the heparinoides from their heparanase-inhibiting properties. Compared to heparin and to the LMWHs, the therapeutic margin of the hexasaccharide Iso ATIII is greatly increased and makes it potentially useable as an anti-metastatic agent. Therefore, the present invention relates to its use as such and the preparation of the hexasaccharide Iso ATIII alone or as a mixture with other hexasaccharides derived from the controlled depolymerization of heparin with heparinase 1. In addition, the present invention relates to the process of depolymerizing heparin with heparinase 1 until its aXa activity is extinguished, and the use of this mixture as an anti-metastatic agent. This will, in this case, be a non-antithrombotic and specifically heparanase-inhibiting LMWH.

The present invention relates to the preparation and the use as an anti-metastatic of the following products, isolated or as a mixture:

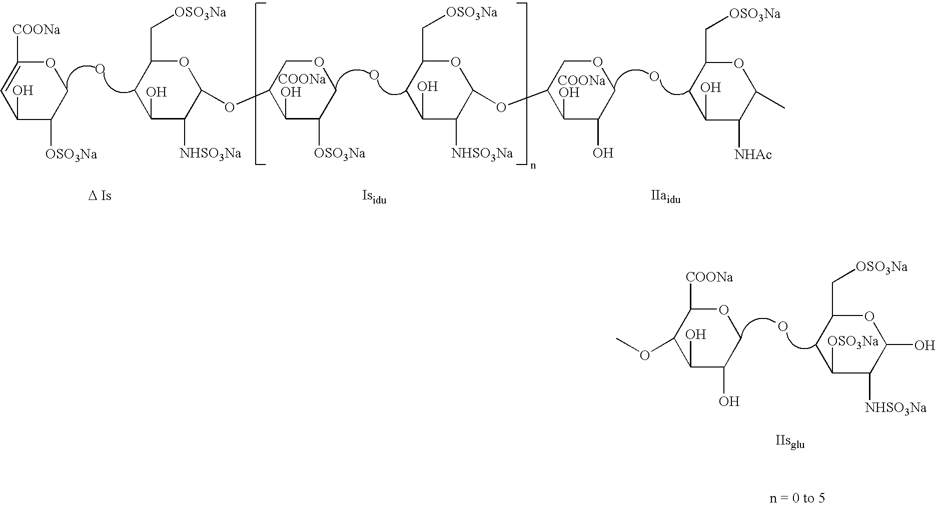

### BRIEF DESCRIPTION OF THE DRAWINGS

FIG. 1: GC monitoring of the heparin depolymerization with heparanase 1.

FIG. 1a: TSK 4000 gel permeation chromatography of a native HS-PG sample.

FIG. 1b: TSK 4000 gel permeation chromatography of a HS-PG sample after heparanase treatment.

FIG. 1c: Inhibition of heparanase activity by unfractionated heparin (UF heparin) (TSK 4000 gel permeation chromatography).

FIG. 2: Inhibition of heparanase activity by unfractionated heparin (UF-heparin).

FIG. 2a: Monitoring of the heparin depolymerization with heparanase by 1 by CTA-SAX.

FIG. 3a: Fractionation of 2.5 g of depolymerised heparin by GC (Biogel P10 column (100×5 cm); Mobile phase: 0.2N NaCl; flow rate 80 ml/h; detection: UV at 250 nm).

FIG. 4: Chromatogram of the hexasaccharide fraction by CTA-SAX.

FIG. 5: Separation of the hexasaccharide fraction by semi-preparative chromatography on a CTA-SAX column (25×2 cm).

FIG. 6: Final chromatogram of the hexasaccharide ΔIs-IIa_{id}-Iis_{glu} after desalification.

FIG. 7: Inhibition of heparanase activity as a function of the concentration of Hexa ISO AT III.

FIG. 8: Identification of the products isolated from the oligosaccharide-rich fractions, for each of the hexasaccharide, octasaccharide and decasaccharide fractions isolated by GC.

### EXPERIMENTAL SECTION

GPC

The gel exclusion chromatography is carried out with 2 TSK Super SW2000 columns (300×4.6 mm) and one TSK Super guard column (35×4.6 mm) (TOSOH BIOSEP). Detection is performed by absorptiometry in the UV range at 232 nm. The mobile phase is 0.1 M ammonium acetate. The injected volume is 5 μl.

CTA-SAX Chromatography

The HPLC monitoring is carried out by the CTA-SAX method. The column used is a 3 μm-particle Hypersil BDS (150×2.1 mm) onto which has been adsorbed cetyl trimethylammonium by percolation of a solution of 1 mM cetyl trimethylammonium hydrogen phosphate in a water/methanol (68/32) v/v mixture at 45° C. at 0.2 ml/min for 4 hours.

The conditions for separation on this type of column are as follows: the temperature of the grafted column is kept at 40° C. An elution gradient, in which solvent A is water adjusted to pH 3 by adding methanesulphonic acid, is effected. Solvent B is a 2N solution of ammonium methanesulphonate adjusted to pH 2.6. The elution gradient is as follows:

| Time | | | Flow rate |
|---|---|---|---|
| (min) | Solvent A | Solvent B | (ml/min) |
| 0 | 99 | 1 | 0.22 |
| 44 | 35 | 65 | 0.22 |
| 74 | 0 | 100 | 0.22 |

The detection used is absorptiometry in the UV range at 232 nm. 202-247 nm is also used as detection specific for acetylated oligosaccharides.
Semi-Preparative Chromatography on CTA-SAX

Chromatography on a 5 μm-particle Hypersil BDS column (250×20 mm) onto which have been grafted cetyl trimethylammonium chains by percolation of a solution of 1 mM cetyl trimethylammonium hydrogen phosphate in a water/methanol (68/32) v/v mixture at 45° C. at 2 ml/min for 4 hours.

The separation is carried out at ambient temperature. An elution gradient is used: solvent A is water brought to pH 2.5 by adding HCl. Solvent B is a 2N NaCl solution adjusted to pH 2.5.

| Time | | | Flow rate |
|---|---|---|---|
| (min) | Solvent A | Solvent B | (ml/min) |
| 0 | 60 | 40 | 10 |
| 44 | 0 | 100 | 10 |

The detection is in the UV range at 232 nm. 100 mg of hexasaccharide fraction can be injected at each separation.

Preparation of the Hexasaccharide Iso ATIII

The hexasaccharide ΔIs-IIa_{id}-IIs_{glu} (hexasaccharide iso ATIII) is obtained by cleavage of the ATIII affinity site of heparin with heparinase 1. The depolymerization of heparin with heparinase 1 is endolytic: it results in a mixture of oligosaccharides unsaturated on their nonreducing end. At the end of the reaction, a mixture of disaccharides, tetrasaccharides and hexasaccharides is obtained. All the most sulphated regions of the heparin are cleaved and converted into disaccharides and into tetrasaccharides. Only the acetylated portions remain in the form of hexasaccharides, and especially the chains of the type -GlcNS(6S or 6OH)-IdoA-GlcNAc(6S or 6OH)-GlcA-GlcNS(3S or 3OH, 6S or 6OH)—

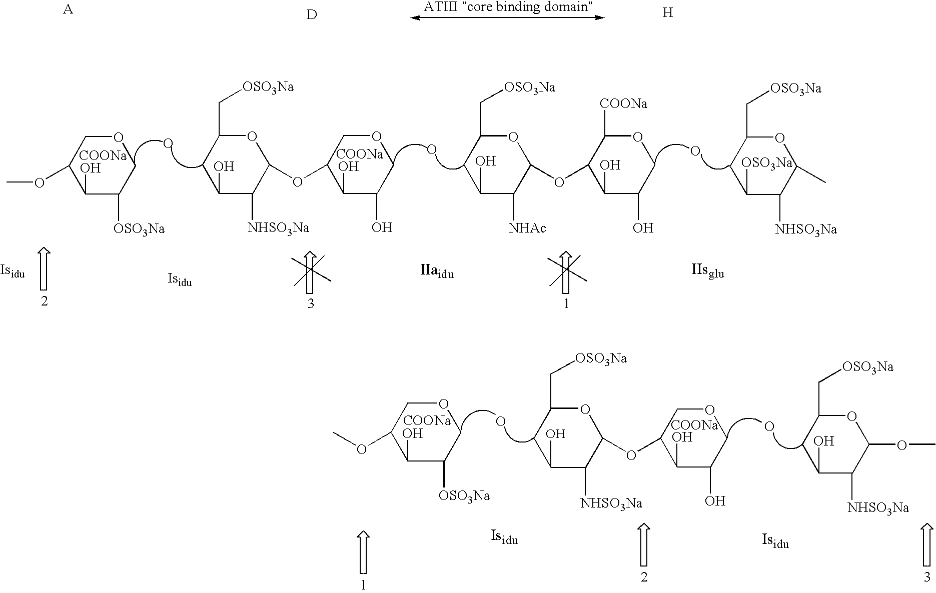

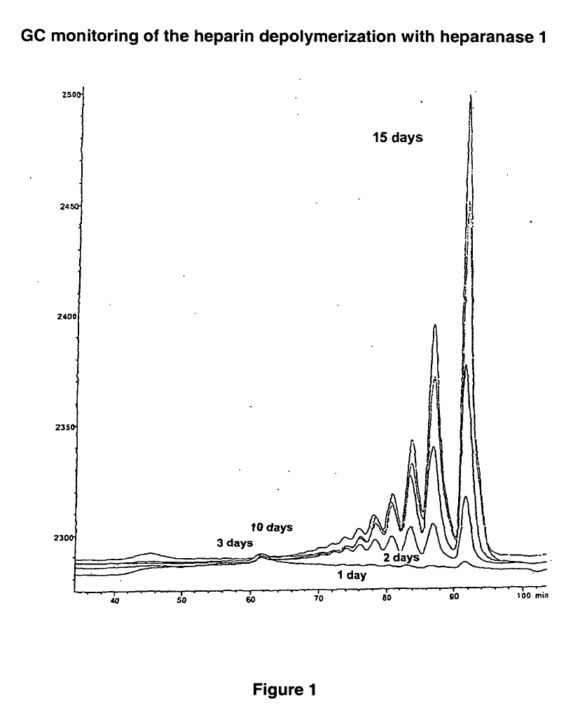

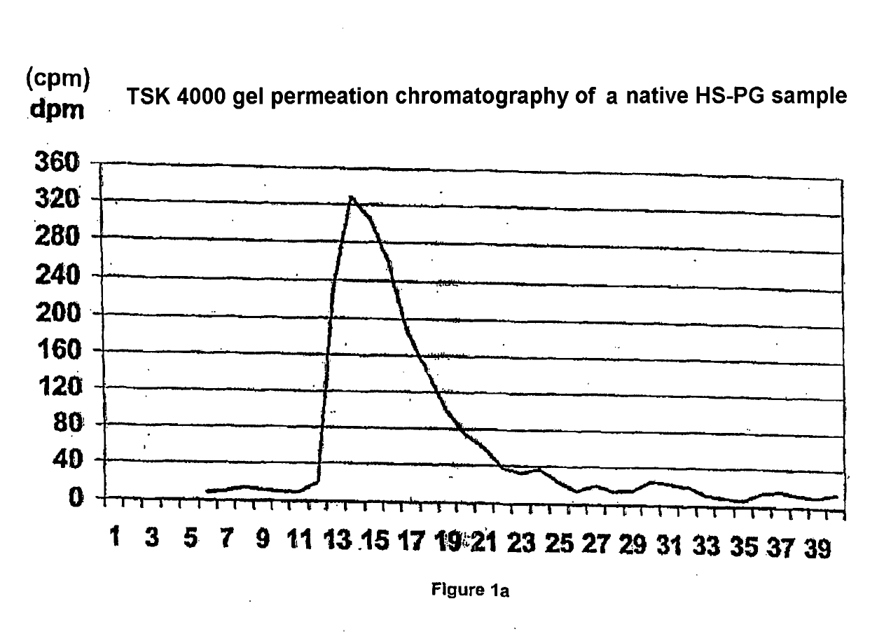

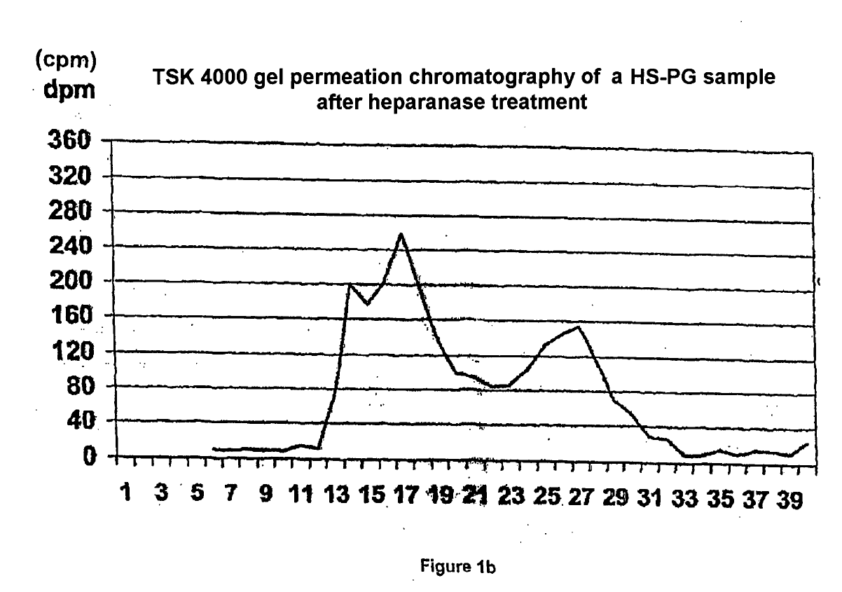

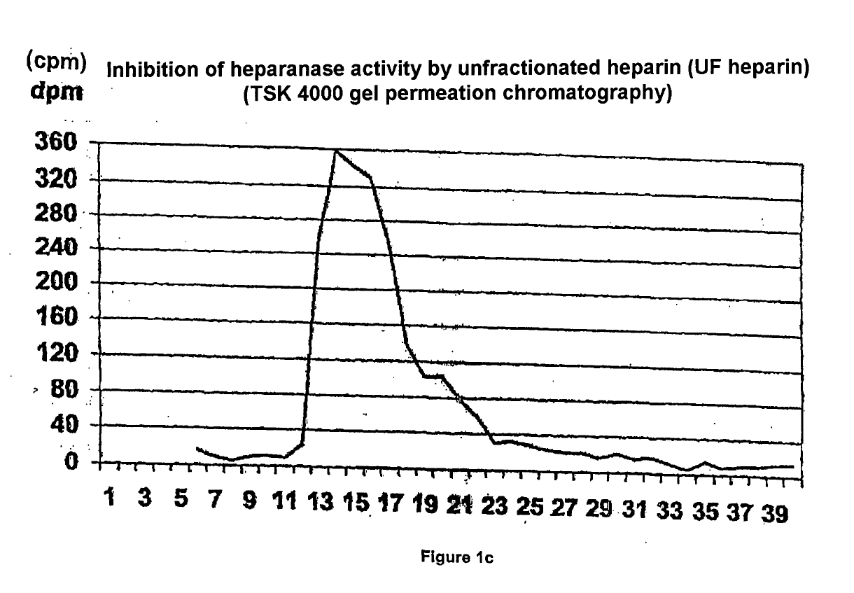

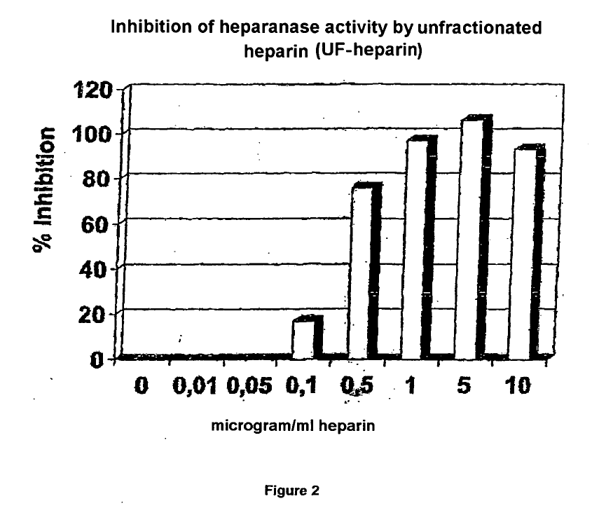

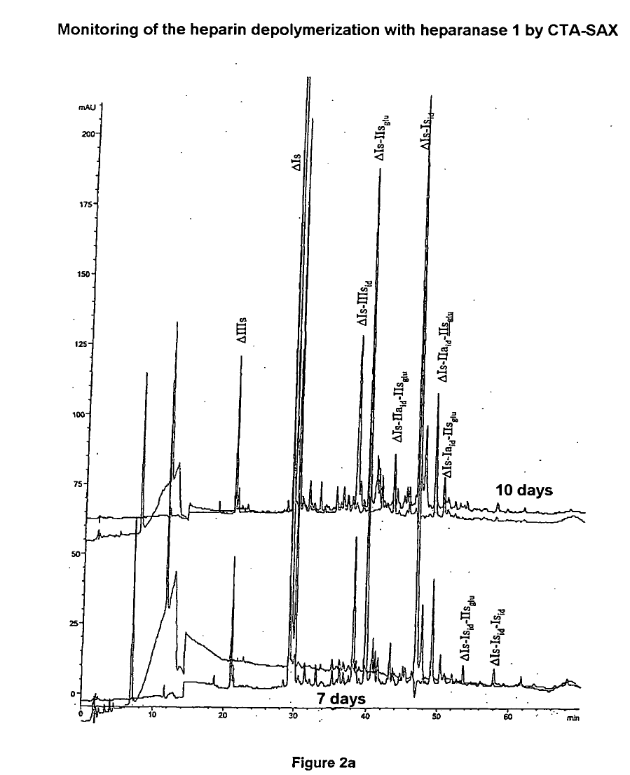

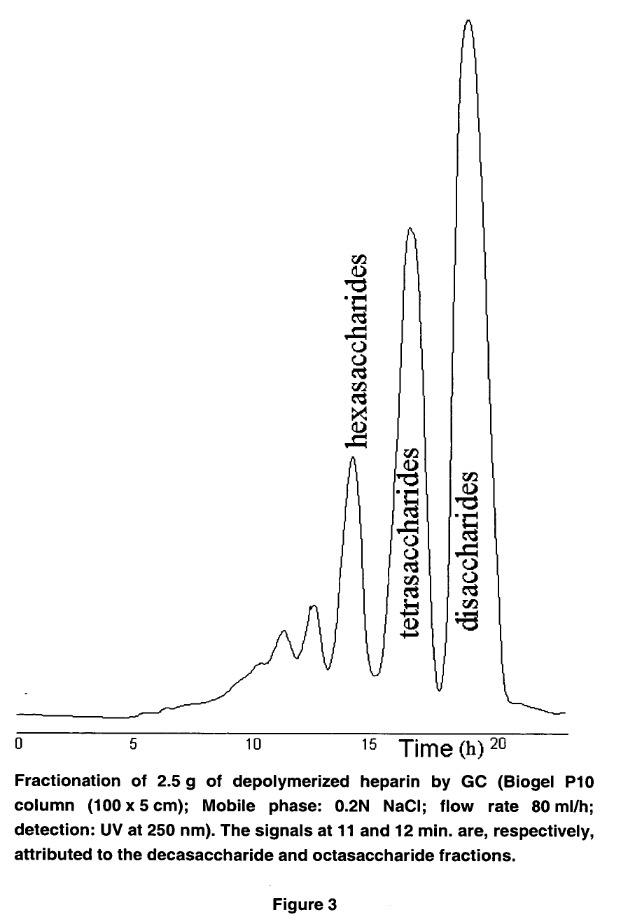

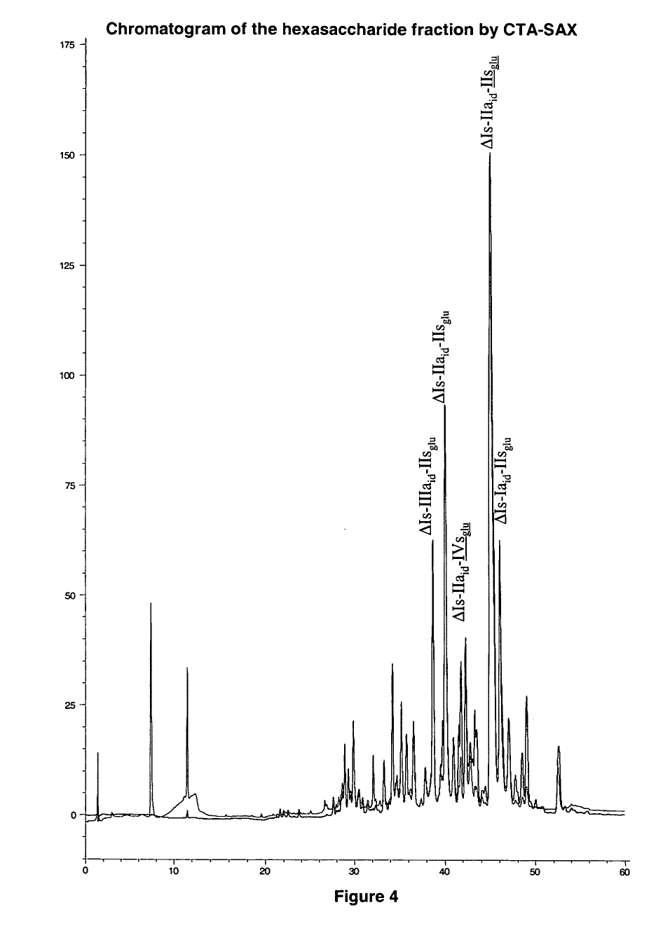

The depolymerization of the heparin takes place under the following conditions: 3 g of heparin from porcine mucous are dissolved in 30 ml of a solution of 0.2M NaCl, 0.02% BSA, 5 mM Na₂HPO₄, adjusted to pH 7. The depolymerization temperature is 16° C. 2 IU of heparinase 1 are initially introduced. After 7 days, an additional unit of heparinase 1 is added. After 15 days, the heparin depolymerization is considered to be finished. The reaction is monitored either by analytical GC on a TSK Super SW 2000 column (FIG. 1), or on a CTA-SAX column (FIG. 2a). The enzyme reaction may be considered to be sufficiently advanced when the proportion of oligosaccharides greater than octasaccharide in size is limited and when the two main sulphated hexasaccharides ΔIs-Is_{id}-Is_{id} and ΔIs-Is_{id}-IIs_{glu} in the mixture have been depolymerized to tetrasaccharides. When the enzyme reaction has finished, the solution is filtered through a 0.2 μm membrane and then injected, in 2 stages, onto a GC column filled with Biogel P10 (Bio Rad), in which a 0.2 N NaCl mobile phase circulates (FIG. 3). The hexasaccharide ΔIs-IIa_{id}-IIs_{glu} is extremely fragile in alkaline medium: it loses its 3-O-sulphated terminal glucosamine and is converted to the pentasaccharide ΔIs-IIa_{id}-GlcA as soon as the pH exceeds 8. It is therefore very important to slightly acidify (pH between 5 and 6) the entire hexasaccharide fraction. The chromatogram for the entire hexasaccharide fraction is given in FIG. 4.

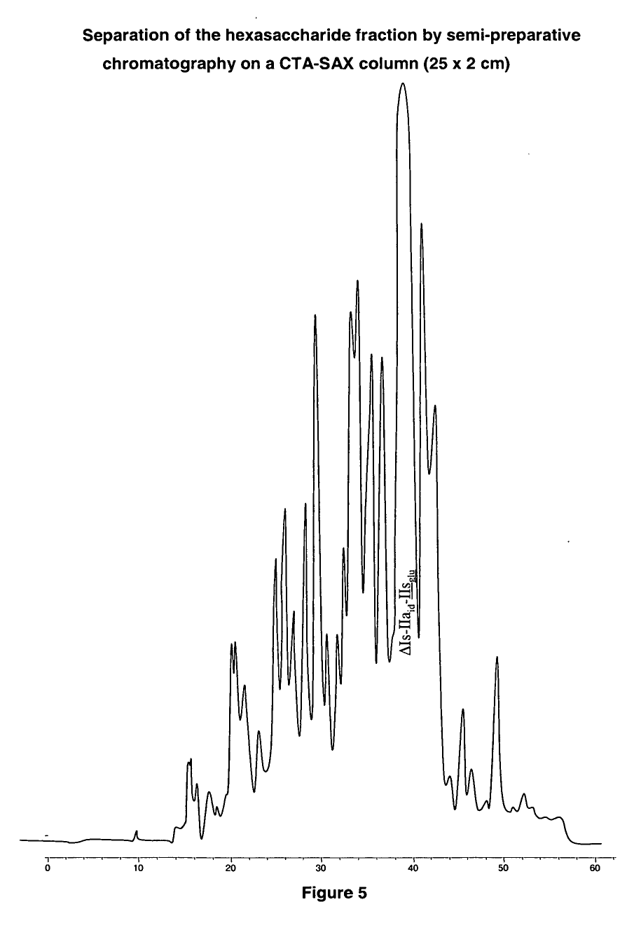

The final phase consists of a semi-preparative separation on a 25×2.1 cm column filled with Hypersil BDS C18 (5 μm) grafted with CTA-SAX (FIG. 5). The fractions are controlled by HPLC. Since the mobile phase used in semi-preparative chromatography is a solution of sodium chloride, it is necessary to prepare a final desalification of the sample. This is carried out in 2 steps. The first step, which removes 95% of the NaCl, consists in re-concentrating the fractions containing the isolated hexasaccharide on a Q-Sepharose High Flow anion exchange phase (Pharmacia) (40×2.6 cm column), by percolating them in the column after they have been diluted 1/10 in water. The hexasaccharide is eluted in a minimal volume (approximately 50 ml) with a 1.5N NaClO₄ solution so as to obtain a solution of hexasaccharide perchlorate.

The second step for final desalification is carried out by injecting the solution of hexasaccharide perchlorate previously obtained onto a Sephadex G10 column (100×7 cm). The monitoring is carried out by UV detection at 232 nm and by means of a conductimeter which makes it possible to detect the salt.

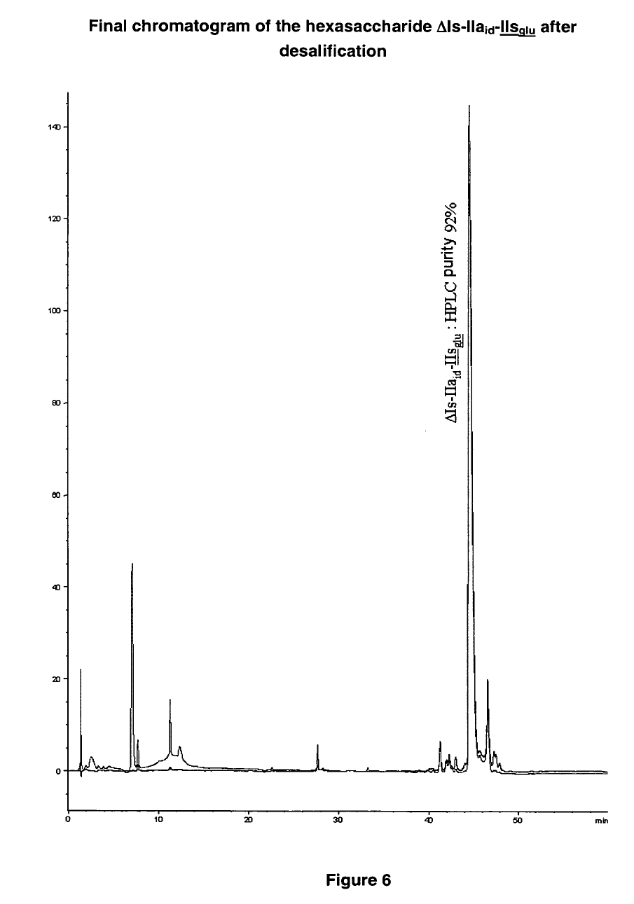

It may prove to be necessary to repeat this operation if the quality of the separation between the hexasaccharide and the perchlorate is insufficient. The hexasaccharide solution is then lyophilized. 108 mg of the hexasaccharide ΔIs-IIa_{id}-IIs_{glu} in the form of the sodium salt are thus obtained. The HPLC purity is 92% (FIG. 6).

Heparanase Biological Activity Assays:

The Evaluation of Hexa Iso ATIII Relative to its Ability to Inhibit Heparanase was Carried Out as Follows:

Radiolabelled heparin/heparan sulphate (HS) is degraded with heparanases, producing low molecular weight HS fragments which can be measured by gel permeation chromatography (FPLC) and counting of the collected fractions by liquid scintillation.

Unfractionated heparin (sodium salt) from porcine intestinal mucosa (grade Ia, 183 USP/mg) was obtained from Sigma Biochemicals (Deisenhofen, Germany).

Heparitinase (HP lyase; (EC 4.2.2.8)) was obtained from Seigaku (Tokyo, Japan).

TSK 4000 comes from Toso Haas and the Sepharose Q columns equipped with precolumns were obtained from Pharmacia/LKB (Freiburg, Germany).

A uterine fibroblast cell line was used to prepare heparan sulphate (proteoglycan) labelled with 35-S by metabolic labelling. It has been shown that this cell line produces relatively large amounts of various heparan sulphate proteoglycans (HS-PGs), such as syndecans and glypican (Drzeniek et al., Blood 93:2884-2897, 1999).

The labelling is carried out by incubating the cells, with a cell density of approximately 1×10⁶ cells/ml, in the presence of 35-S-sulphate at 33 μCi/ml in the tissue culture medium for 24 hours. The supernatants are then collected and a protease inhibitor, PMSF (phenylmethylsulfonyl fluoride) (1 mmol/l), is added. The HS-PGs are purified by anion exchange chromatography on Sepharose Q, elimination of the chondroitin sulphate and dermatan sulphate (proteoglycans) not being necessary since the sample contains a relatively large amount of heparan sulphate proteoglycans, and also due to the specificity of the heparanase enzyme.

The heparanase was isolated from human peripheral blood leukocytes (PBLs, buffy coats), enriched with polymorphonuclear cells (PMNs) by ficoll gradient procedures. The concentration of the isolated PMNs is adjusted to 2.5×10⁷ cells/ml and incubated for 1 hour at 4° C. The supernatants containing the heparanase are then collected, the pH is adjusted to 6.2 (20 mM of citrate-phosphate buffer) and they are either used immediately or stored frozen in aliquots at −20° C.

200 μl of 35-S-labelled heparan sulphate (proteoglycans) adjusted to approximately 2200 cpm/ml (cpm=counts per minute) are incubated at 37° C. for 18 hours with 1 μl of PMN supernatant containing the heparanase. 200 μl of the mixture obtained above are sampled on a TSK 4000 gel permeation chromatography column (FPLC), and the fractions are collected and analyzed by liquid scintillation counting. The degradation was measured according to the following formula:
% degradation=[[Σcounts (cpm) fract. 20-33 (HEP)−Σcounts (cpm) fract. 20-33 (CONT)]/[total counts (cpm) fract. 12-33 (CONT)]]×100

For example, the percentage degradation is calculated as follows: the sum of the counts (cpm) in fractions 20-33 of the sample after treatment with the heparanase, minus the background noise count (cpm) (fractions 20-33) of the control sample, is divided by the total counts (fractions 12-33) applied to the column. Correction factors were used to standardize the total counts of various rounds of chromatography, at 2200 counts/cpm. The results are given as percentage degradation. In the inhibition assays, the degradation of the control sample (with heparanase) was fixed at 100% (degradation), and the values of % inhibition were calculated on this basis. A correction for the sulphatase activity is not necessary since no sulphatase activity could be detected.

The following heparanase inhibitors: unfractionated heparin (UF-H) and Hexa Iso ATIII were assayed via the protocol described above at three different concentrations. The comparison was made on a weight basis. The data are expressed as percentage inhibition of the heparanase activity.

Results

Firstly, the heparanase assay was optimized for the needs of this study. For practical reasons, the incubation time in the degradation assay was established at 18 hours. Depending on the efficiency of labelling and the content of heparan sulphate (proteoglycans), the total heparan sulphate (proteoglycans) count was fixed at approximately 2200 cpm per sample, so as to make it possible to carry out all the assays with one batch of heparan sulphate (proteoglycan). FIG. 1a shows the TSK 4000 gel permeation chromatography of a native sample. FIG. 1b shows the heparanase-induced shift in the molecular distribution of the sample. The amount of heparanase which allows degradation of approximately 80% of heparan sulphate proteoglycan is then determined (the sample containing approximately 35% of heparan sulphate proteoglycans and approximately 65% of chondroitin/dermatan sulphate proteoglycans). Consequently, a degradation in the range of 10-80% is relatively linear and is acceptable for determining the effect of the inhibitors. FIG. 1c shows the effect of unfractionated heparin (UFH) at 1 μg/ml on the heparanase activity, with an inhibition of 97.3%.

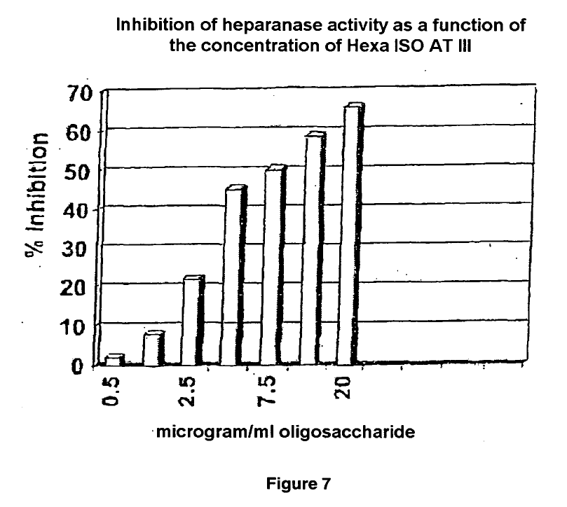

After having determined the assay conditions, the effect of unfractionated heparin (UFH) derived from porcine intestinal mucosa was measured. FIG. 2 shows a dose-dependant inhibition. Virtually complete inhibition of the heparanase activity was observed at a concentration of unfractionated heparin (UFH) of 1 μg/ml (final concentration). FIG. 7 shows the dose-dependant inhibition by Hexa Iso ATIII. On the basis of these data, it may be concluded that Hexa iso ATIII exhibits a strong heparanase-inhibiting activity.

The content of the following publications is integrated herein by way of reference:
C. R. Parish, et al., Biochim. Biophys. Acta 1471 (2001) 99-108
M. Bartlett et al., Immunol. Cell Biol. 73 (1995) 113-124
I. Vlodavsky et al., IMAJ 2 (2000) 37-45
Y. Matzner, et al., J. Clin. Invest 76 (1985), 1306-1313
Z. Drzeniek, et al., Blood (1999) 2884-2897

Other oligosaccharide-rich fractions can be isolated from the product of degradation of heparin by heparinase 1. Thus, in the case of hexasaccharides, a single CTA-SAX chromatographic purification is sufficient. This method uses a Hypersil BDS (250×20 mm) column, 5 μm particles, onto which cetyltrimethylammonium chains have been grafted by percolation of a 1 mM solution of cetyltrimethylammonium hydrogen phosphate in a water-methanol mixture (68-32) v/v at 45° C. at 2 ml/min for 4 hours.

The separation is carried out at ambient temperature. An elution gradient is used: solvent A is water brought to pH 2.5 by the addition of HCl. Solvent B is a 2N solution of NaCl adjusted to pH 2.5.

| Time | | | Flow rate |
|---|---|---|---|
| (min) | Solvent A | Solvent B | (ml/min) |
| 0 | 60 | 40 | 10 |
| 44 | 0 | 100 | 10 |

Detection is in the UV range at 232 nm. 100 mg of hexasaccharide fraction can be injected at each separation.

The purification of the octasaccharide and decasaccharide fractions is more complex than that of the hexasaccharide fractions. In general, it requires an additional purification on an IonPac ®AS11 column (250×20 mm) (Dionex). The separation is carried out at ambient temperature. An elution gradient is used. Solvent A is water brought to pH 3 by the addition of perchloric acid. Solvent B is a 1 M solution of NaClO₄ adjusted to pH 3.

| Time | | | Flow rate |
|---|---|---|---|
| (min) | Solvent A | Solvent B | (ml/min) |
| 0 | 99 | 1 | 20 |
| 80 | 40 | 60 | 20 |

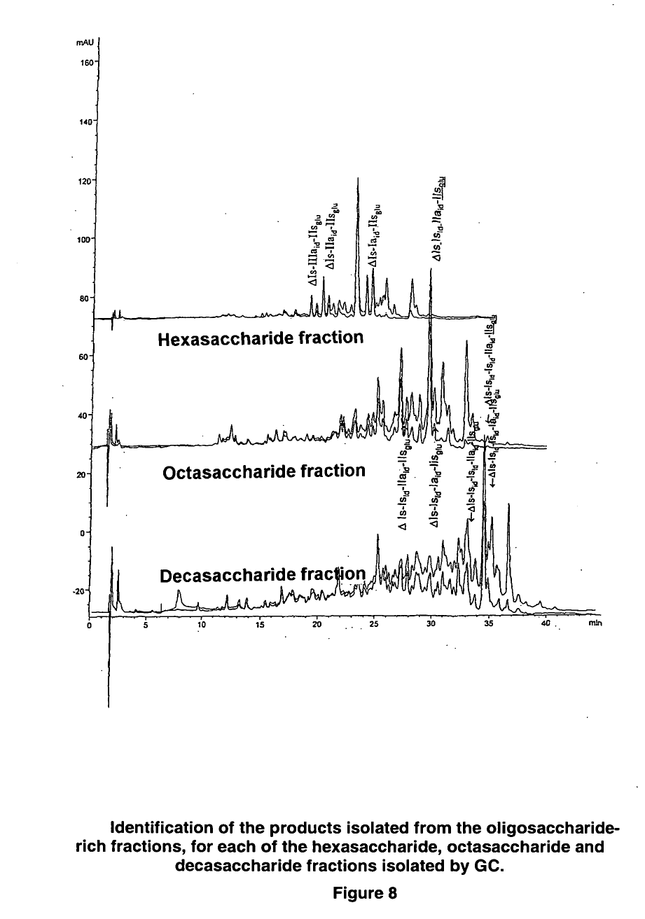

FIG. 8 makes it possible to identify the products isolated from the oligosaccharide-rich fractions, for each of the hexasaccharide, octasaccharide and decasaccharide fractions isolated by GC.

Evaluation of the Activity of the Heparanase Inhibitors in an Enzymatic System

The activity of the heparanase is demonstrated by virtue of its ability to degrade fondaparinux. The concentration of fondaparinux is determined by virtue of its anti-factor Xa activity.

A. Materials and Methods

The heparanase is produced by Sanofi-Synthelabo (Labège, France).

The reagents for assaying factor Xa are sold by Chromogénix (Montpellier, France).

Increasing concentrations of a compound according to the invention, heparanase inhibitor (variable dilutions: from 1 nM to 10 μM), are mixed with a fixed concentration of heparanase (for each batch, preliminary experiments make it possible to determine the enzymatic activity sufficient for degradation of 0.45 μg/ml of fondaparinux added). After 5 minutes at 37° C., the mixture is brought into contact with the fondaparinux and left at 37° C. for 1 hour. The reaction is stopped by heating at 95° C. for 5 minutes. The residual fondaparinux concentration is finally measured by adding factor Xa and its specific chromogenic substrate (Ref. S2222).

The Various Mixtures are Prepared According to the Following Procedure:

a) Reaction Mixture

50 μl of sodium acetate buffer (0.2 M, pH 4.2) are mixed with 50 μl of fondaparinux (0.45 μg/ml) and 59 μl of a heparanase solution. The mixture is incubated for 1 hour at 37° C. and then for 5 minutes at 95° C. The pH thus goes from 4.2 to 7. 100 μl of the reaction mixture are then mixed with 50 μl of 50 mM Tris buffer containing 175 mM NaCl and 75 mM EDTA, pH 14.

The anti-factor Xa activity of the fondaparinux is measured in the following way:

b) Assaying of the Anti-Factor Xa Activity of Fondaparinux

100 μl of the solution obtained in step a) are mixed with 100 μl of AT (0.5 μg/ml). The mixture is kept at 37° C. for 2 minutes and 100 μl of factor Xa (7 nkat/ml) are then added. The mixture is kept at 37° C. for 2 minutes and 100 μl of chromogenic substrate (Ref.: S2222) (1 mM) are then added. The mixture is kept at 37° C. for 2 minutes and then 100 μl of acetic acid (50%) are added.

The optical density is read at 405 nm.

A percentage inhibition is determined relative to the control without inhibitor. A percentage inhibition curve makes it possible to calculate an IC₅₀.

B. Results

| | | | % |
|---|---|---|---|
| Product | Structure | Concentration (M) | inhibition |
| Hexa Iso ATIII | ΔIs-IIa-IIs | 3.00E−5 | 48.5 |
| (Ia) | ΔIs-IIa-IIs | 1.00E−4 | 53.8 |
| (Ib) | ΔIs-Is-IIa-IIs | 1.00E−5 | 59.9 |
| (Ic) | ΔIs-Is-IIa-IIs | 3.00E−6 | 59.2 |
| (Id) | ΔIs-Is-Ia-IIs | 3.00E−5 | 53.9 |
| (Ie) | ΔIs-Is—Is-IIs | 3.00E−5 | 59.1 |
| (If) | ΔIs-Is—Is-IIa-IIs | 3.00E−6 | 58.4 |
| (Ig) | ΔIs-Is—Is-IIa-IIs | 1.00E−4 | 55.8 |
| (Ih) | ΔIs-Is—Is-Ia-IIs | 3.00E−5 | 55.5 |
| (Ij) | ΔIs-Is—Is—Is-IIs | 3.00E−5 | 48.4 |
| (Im) | ΔIs-Ia-IIs | 3.00E−5 | 54.1 |
| Hexasaccharide | | 3.00E−5 | 55.8 |
| fraction | | | |
| Octasaccharide | | 3.00E−6 | 50.7 |
| fraction | | | |
| Decasaccharide | | 3.00E−6 | 55.6 |
| fraction | | | |
| Crude after | | 3.00E−6 | 53.0 |
| depolymerization | | | |

What is claimed is:

1. A process for depolymerizing a polysaccharide with anti-thrombotic properties, for obtaining a product having anti-cancer properties, comprising a step in which the polysaccharide is depolymerized with heparinase 1 until its anti-thrombotic activity is essentially extinguished (<35 IU/mg), wherein the depolymerization is carried out at a temperature of between 10 and 20° C.

2. The process according to claim 1, wherein the polysaccharide is a heparin.

3. The process according to claim 1, wherein the depolymerization is pursued until the mixture comprises a hexasaccharide fraction essentially free of sulphated hexasaccharides ΔIs-Is_{id}-Is_{id} and ΔIs-Is_{id}-IIs_{glu}.

4. The process according to claim 1, wherein the depolymerization is pursued until an average molecular mass of less than 5000 Da is attained.

5. The process according to claim 1, wherein the depolymerization is pursued until an average molecular mass of less than 3000 Da is attained.

6. The process according to claim 1, further comprising a step in which the product of depolymerization of the polysaccharide is purified by gel permeation chromatography at a pH below 8 and above 5.

7. The process according to claim 6, further comprising a step of purification by high performance liquid chromatography (HPLC) in which a stationary phase is a reverse phase which is (i) C18-grafted and (ii) grafted with cetyl trimethylammonium (CTA-SAX).

8. The process according to claim 7, further comprising a desalification step.

9. The process according to claim 8, in which the desalification step comprises the use of a mobile phase containing an electrolyte in aqueous solution, essentially transparent between 200 and 250 nm.

10. The process according to claim 9, wherein the electrolyte is chosen from perchlorates, methanesulphonates or phosphates of alkali metals.

11. The process according to claim 10, wherein the desalification is carried out using an anion exchange resin.

12. The process according to claim 8, further comprising a second desalification step using a molecular exclusion gel.

13. The product obtained by the process according to claim 1.

14. The product of formula (I) 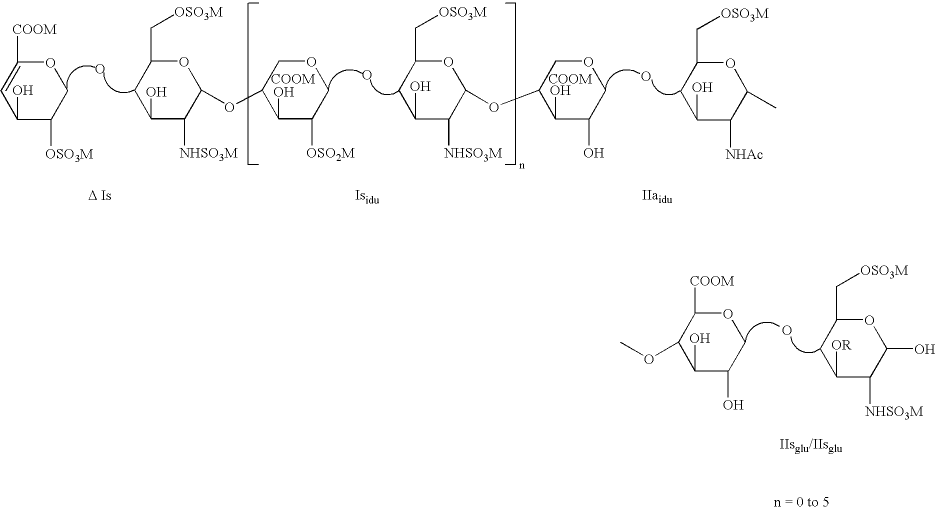
in which:
R is chosen from H and SO₃M, and
M is chosen from H, Li, Na and K;
with the exception of the product for which n=0, R=SO₃M and M=Na.

15. The product according to claim 14, wherein M is chosen from Li, Na and K.

16. The product according to claim 14, wherein n=0.

17. The product according to claim 16, wherein M=Na.

18. The product according to claim 17, of formula (Ia):

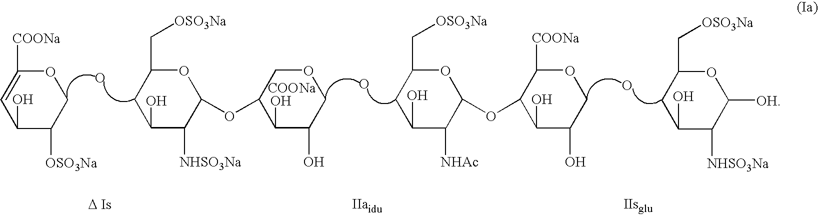

19. The product according to claim 14, of formula (Ib):

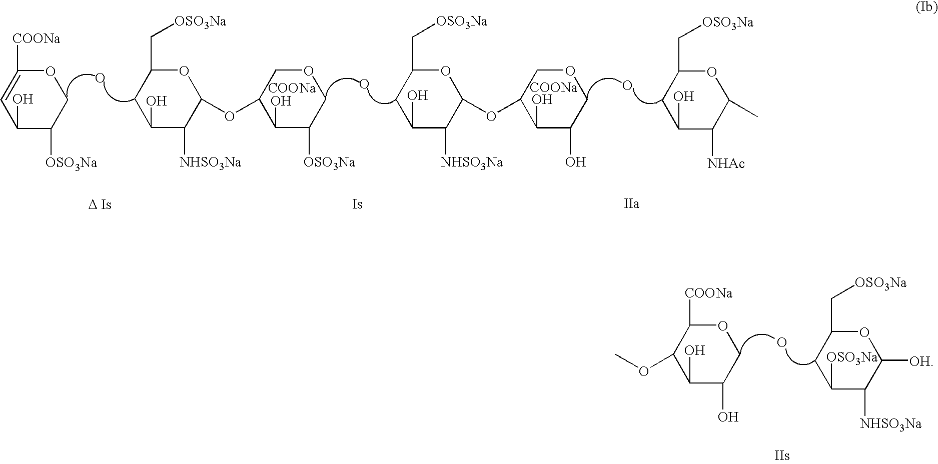

20. The product according to claim 14, of formula (Ic):

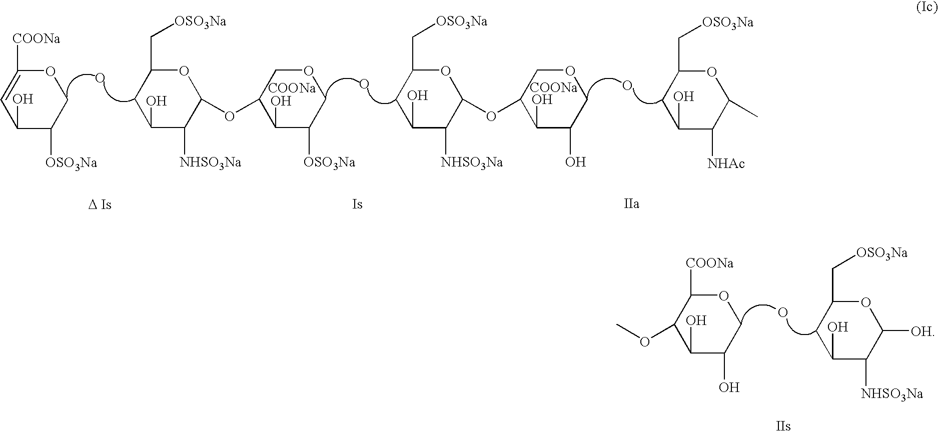

21. The product of formula (Id):

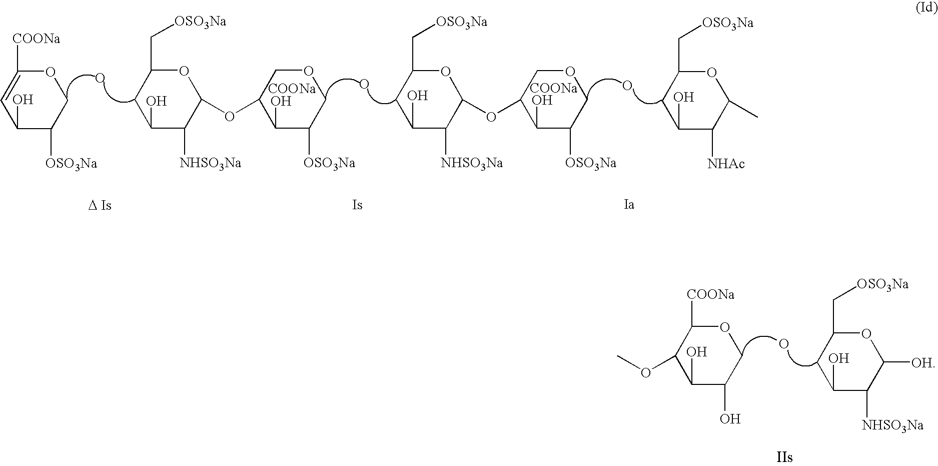

22. The product of formula (Ie):

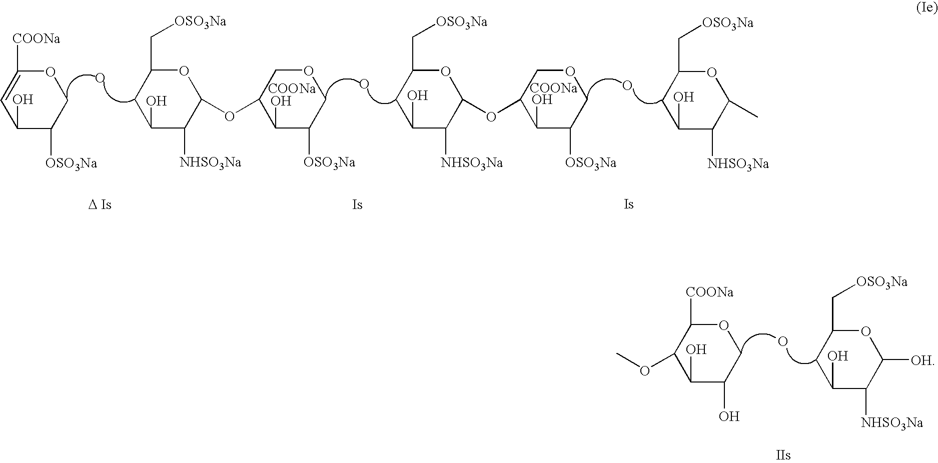

23. The product according to claim 14, of formula (If):

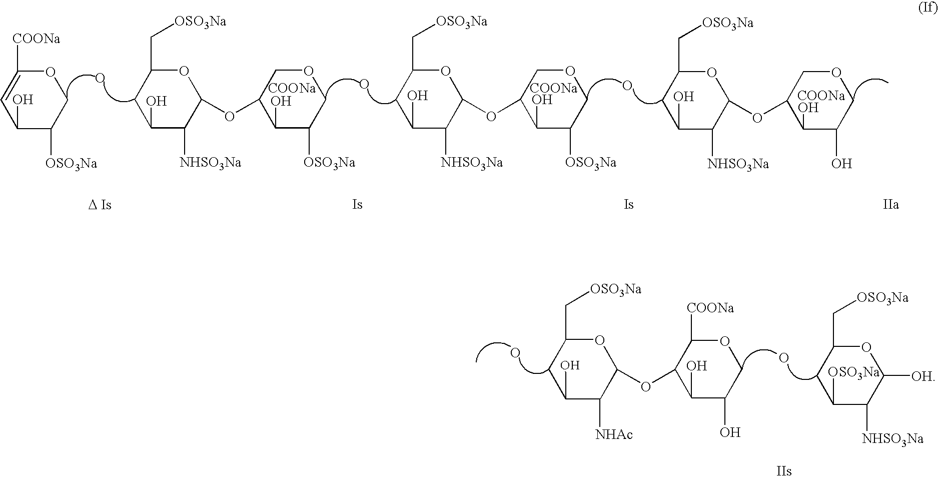

24. The product according to claim 14, of formula (Ig):

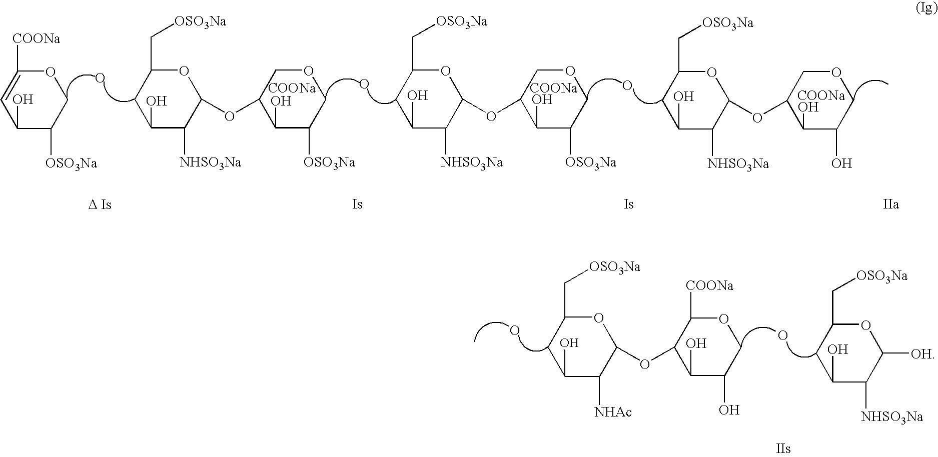

25. The product of formula (Ih):

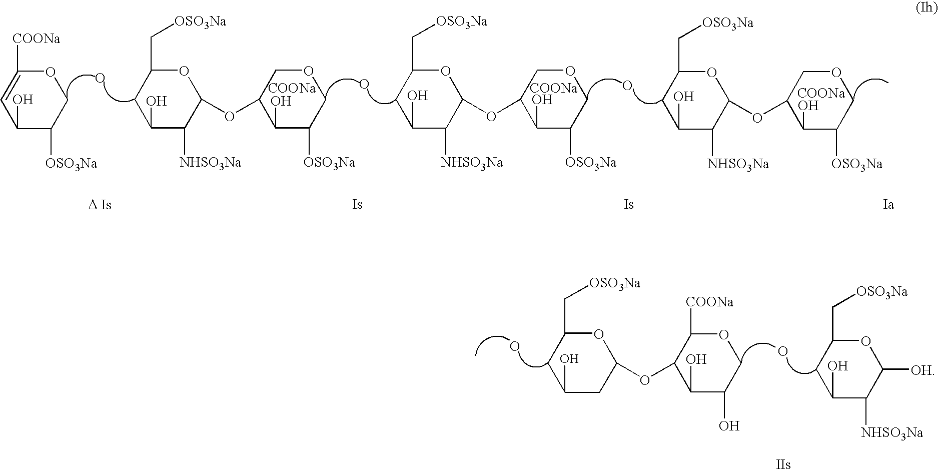

26. The product of formula (Ij):

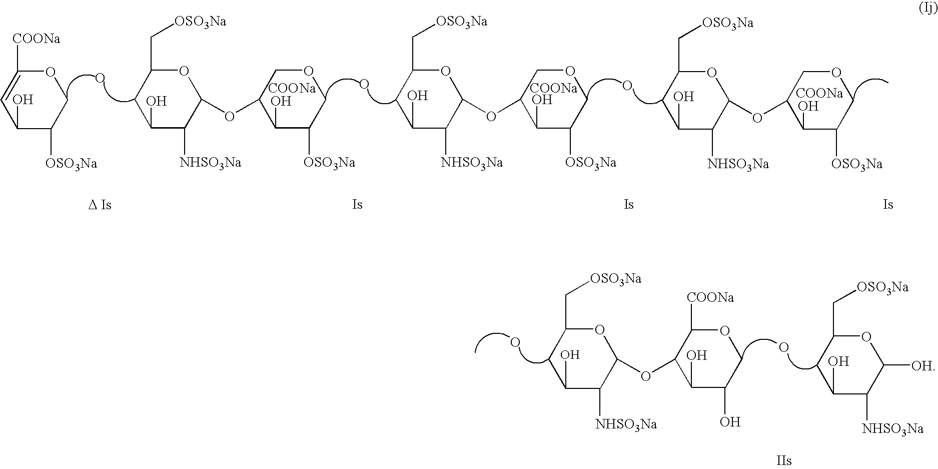

27. The product according to claim 14, of formula (Ik):

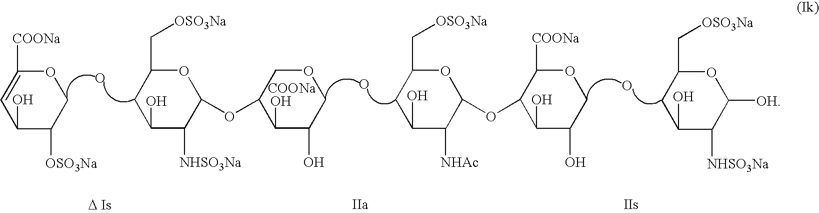

28. The product of formula (Im):

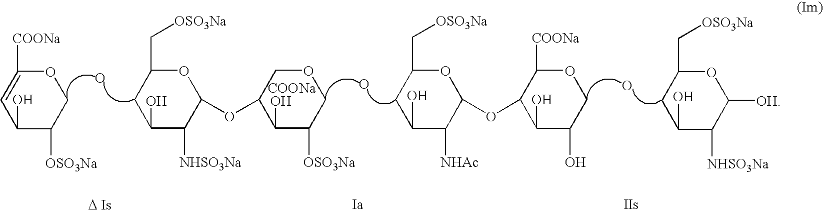

29. A method of inhibiting heparanase which comprises administering to a patient an effective amount of a product of formula (I): 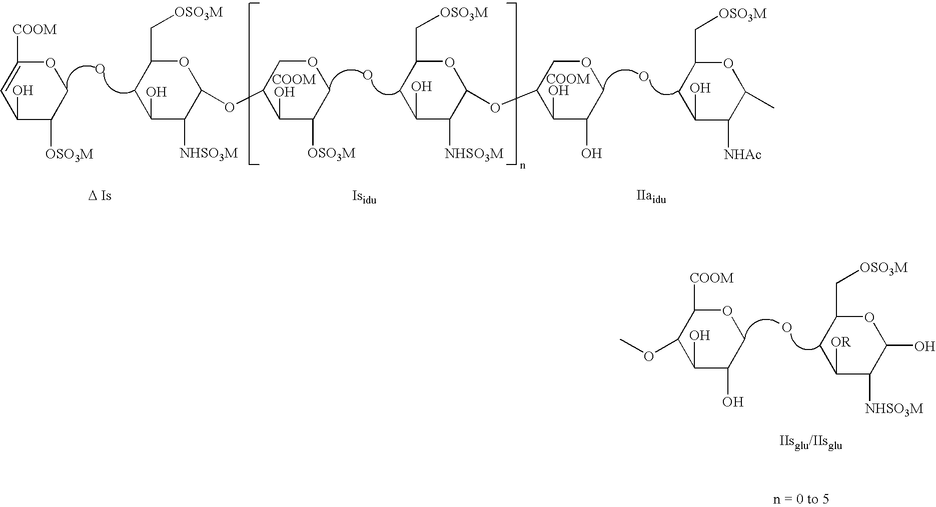
in which:
R is chosen from H and SO₃M, and
M is chosen from H, Li, Na and K.

30. The method according to claim 29, wherein the product is:

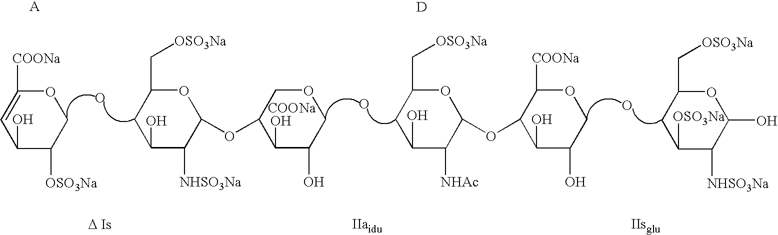

31. A method of inhibiting heparanase which comprises administering to a patient an effective amount of a product selected from the group consisting of:

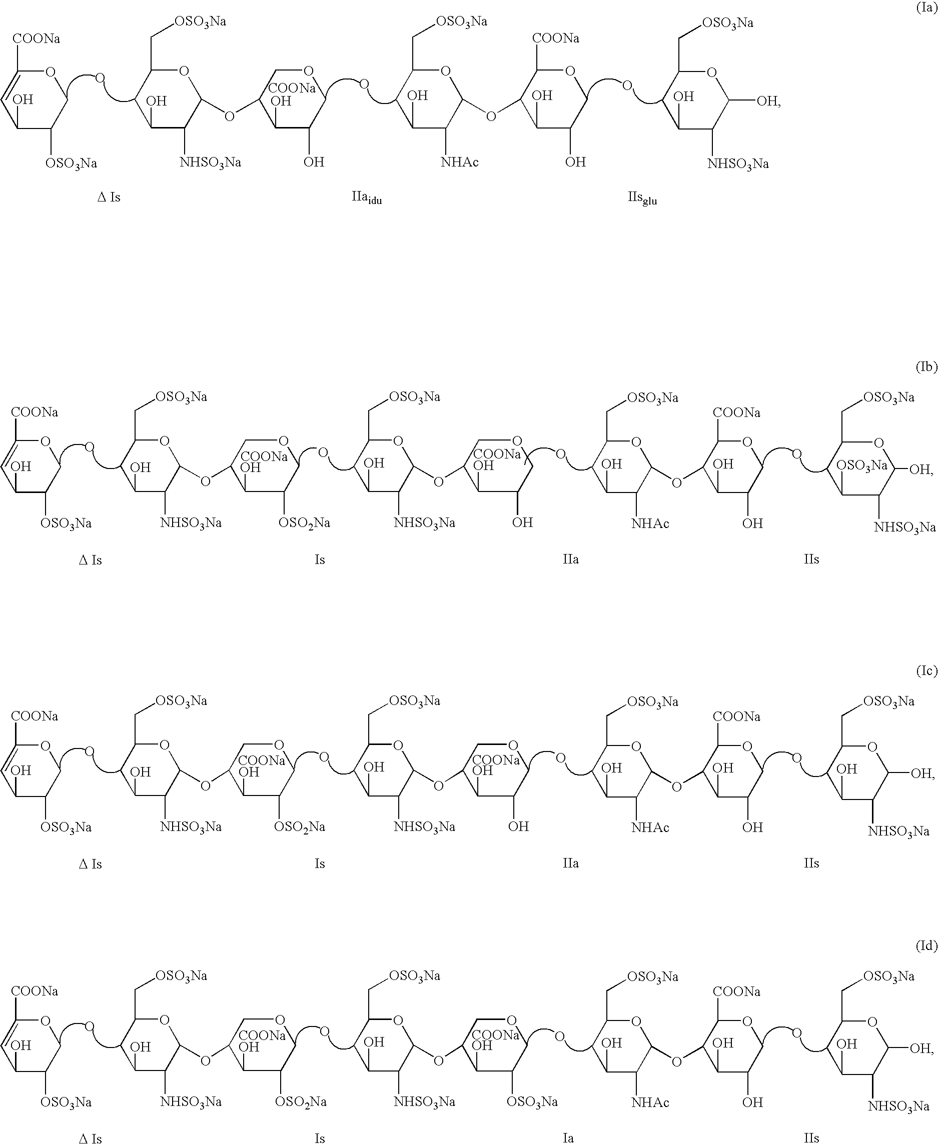

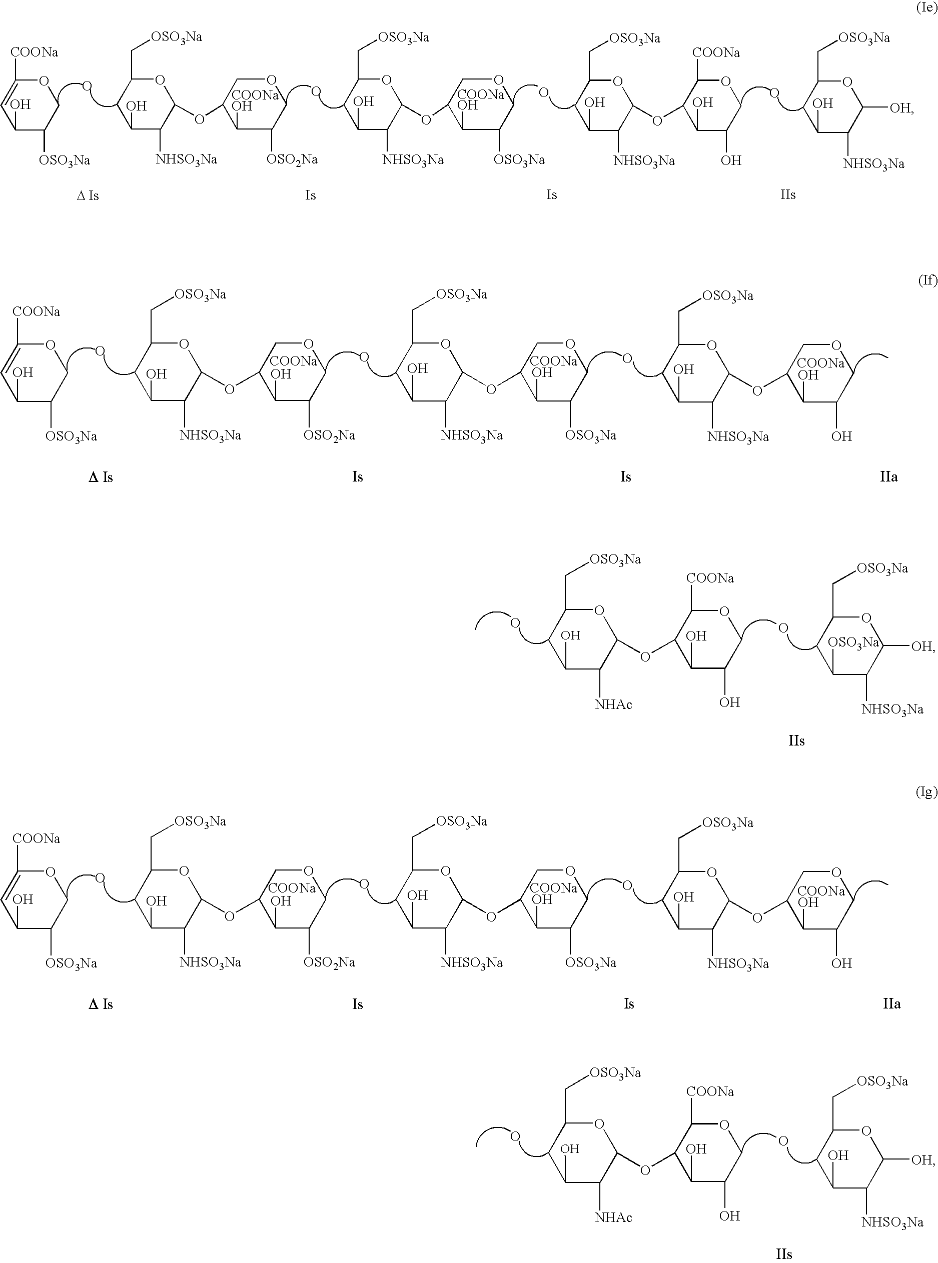

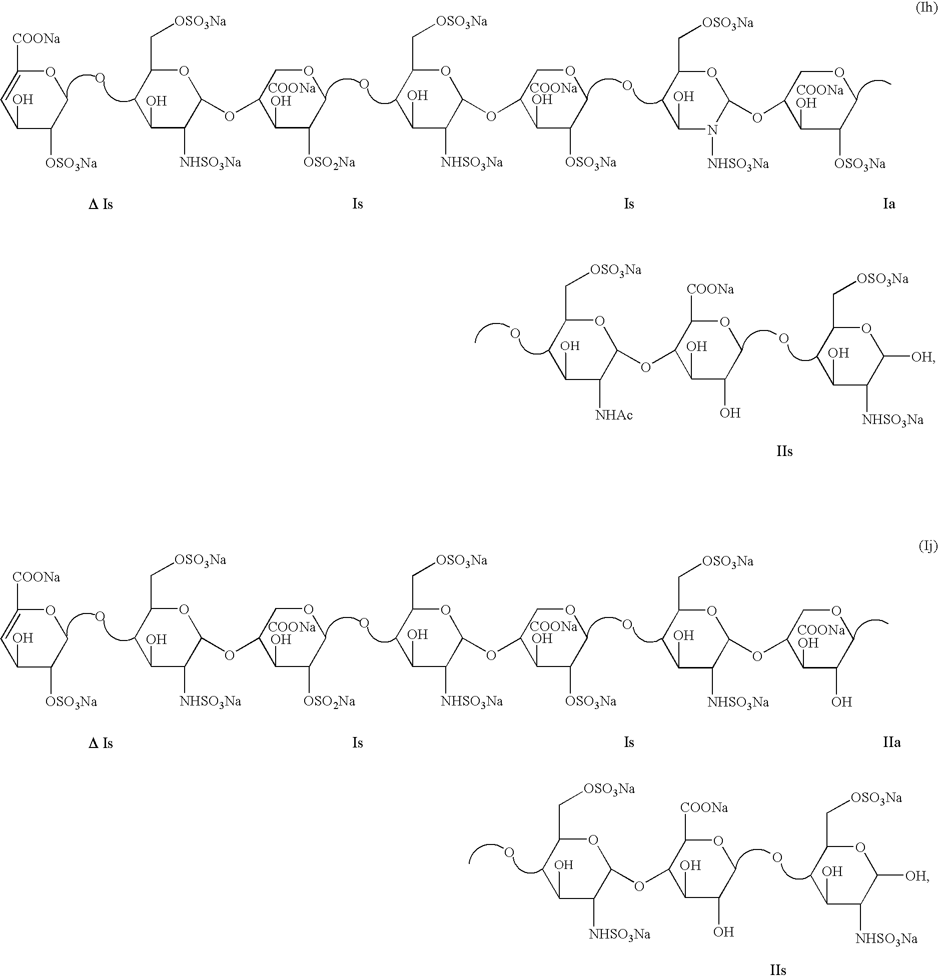

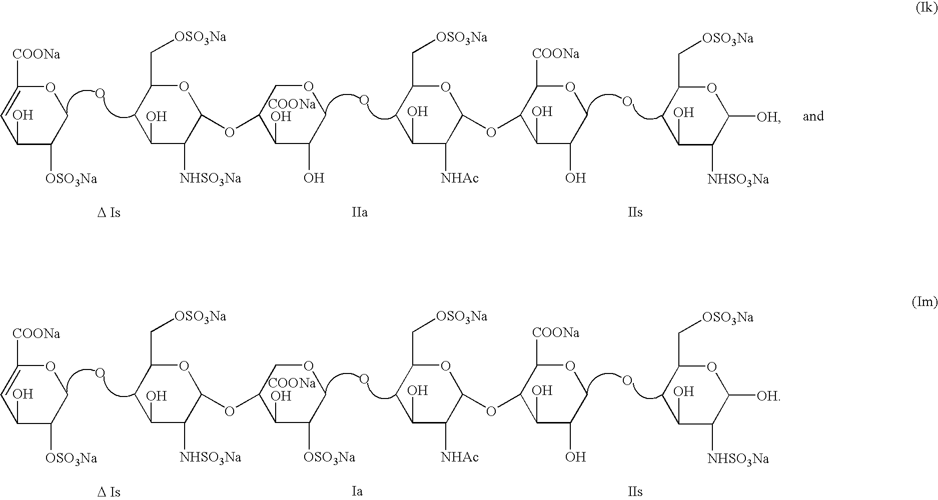

32. A method of modulating cell proliferation, which comprises administering to a patient an effective amount of the product according to claim 14.

33. The method according to claim 32, wherein the cell proliferation is related to a metastatic process.

34. A method of treating cancer, which comprises administering to a patient an effective amount of the product according to claim 14.

35. The method according to claim 34, wherein the treatment prevents or inhibits the formation of metastases

36. The method according to claim 34, wherein the product is administered at an early stage of the disease.

37. The method according to claim 34, wherein the cancer is breast cancer, lung cancer, prostate cancer, colon cancer or pancreatic cancer.

38. A method of modulating cell proliferation, which comprises administering to a patient an effective amount of a product according to claim 18.

39. The method according to claim 38, wherein the cell proliferation is related to a metastatic process.

40. A method of treating cancer, which comprises administering to a patient an effective amount of the product according to claim 18.

41. The method according to claim 40, wherein the treatment prevents or inhibits the formation of metastases.

42. The method according to claim 40, wherein the product is administered at an early stage of the disease.

43. The method according to claim 40, wherein the cancer is breast cancer, lung cancer, prostate cancer, colon cancer or pancreatic cancer.

44. A method of modulating cell proliferation, which comprises administering to a patient an effective amount of a product according to claim 14 in combination with a second anticancer product.

45. The method according to claim 44, wherein the second anticancer product is cytotoxic.

46. The method according to claim 44, wherein the second anticancer product is chosen from the group consisting of platinum derivatives, taxoids, purine base or pyrimidine base derivatives, vincas, mustards, condensed aromatic heterocycles, ellipticine, camptothecins, topotecan, combretastatins, and colchicine derivatives.

47. The method according to claim 44, wherein the second anticancer product is docetaxel, oxaliplatin or irinotecan.

48. A method of modulating cell proliferation, which comprises administering to a patient an effective amount of a product according to claim 18 in combination with a second anticancer product.

49. The method according to claim 48, wherein the second anticancer product is cytotoxic.

50. The method according to claim 48, wherein the second anticancer product is chosen from the group consisting of platinum derivatives, taxoids, purine base or pyrimidine base derivatives, vincas, mustards, condensed aromatic heterocycles, ellipticine, camptothecins, topotecan, combretastatins, and colchicine derivatives.

51. The method according to claim 48, wherein the second anticancer product is docetaxel, oxaliplatin or irinotecan.
